(19) United States
(12) Patent Application Publication
(10) Pub. No.: US 2007/0000072 A1
(43) Pub. Date: Jan. 4, 2007

(54) Care hair coloran

(76) Inventors: Johann Aeby, Marly (CH); Herbert Mager, Marly (CH); Hans Rudolf Murner, St. Ursen (CH); Magali Bourqui, Fout (CH)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/357,581

(22) Filed: Feb. 17, 2006

(30) Foreign Application Priority Data
Feb. 17, 2005 (DE) 102005007296.8

(51) Int. Cl. A61K 8/00

(52) U.S. Cl. 8/405

(57) ABSTRACT

The invention relates to an emulsion-like color carrier mass which comprises (a) water, (b) oxidation color precursors and optionally additionally dyes which attach directly to the hair, (c) at least one nonionic organic thickener with wax-like properties from the group consisting of C12-C24-fatty alcohols and glycerol oleate and mixtures thereof, (d) as nonionic surfactant a mixture of at least one sorbitan fatty acid ester oxyethylated with 4 to 20 mol of ethylene oxide and at least one C12-C24-fatty alcohol oxyethylated with 30 to 70 mol of ethylene oxide, and (e) cosmetically customary cationic resins, and (f) is free from anionic and cationic surfactants.

The present application relates to a special dye carrier mass based on a care cream and to a composition for the oxidative coloring of hair obtained by mixing this color carrier mass with an oxidizing agent.

Oxidation hair colorants are a central constituent of any cosmetic product range used for treating the hair. They consist of two components which are mixed shortly prior to use and then applied to the hair to be colored.

The first component is referred to as dye carrier mass and comprises the dyes, while the second component comprises an oxidizing agent, in particular hydrogen peroxide.

The emulsion-like dye carrier masses known hitherto have a series of disadvantages, in particular with regard to the hair care effect, the resistance of this effect to shampoo and compatibility with the skin. It is therefore an aim of the present invention to overcome these disadvantages, the intention being to dispense with the use of the cationic emulsifiers usually used as care additives for the purpose of improving skin compatibility and color evenness.

Surprisingly, it has now been found that the abovementioned requirements are satisfied in an excellent manner if a special combination of certain nonionogenic emulsifiers, nonionogenic, wax-like thickeners and cationic resins is used, the composition being free from anionic and cationic emulsifiers.

The present invention therefore relates to an emulsion-like dye carrier mass comprising (a) water, (b) oxidation color precursors and optionally additionally dyes which attach directly to the hair and (c) customary cosmetic cationic resins, characterized in that it comprises (d) at least one nonionic organic thickener with wax-like properties from the group consisting of C12-C24-fatty alcohols and glycerol oleate and mixtures thereof, (e) as nonionic surfactant a mixture of at least one sorbitan fatty acid ester oxyethylated with 4 to 20 mol of ethylene oxide and at least one C12-C24-fatty alcohol oxyethylated with 30 to 70 mol of ethylene oxide, and (f) is free from anionic and cationic surfactants

Besides the nonionogenic surfactants used, amphoteric surfactants can also optionally be used. However, preference is given to embodiments without the addition of amphoteric surfactants.

Preferably, the present new color carrier mass is free from nonionic and anionic thickening polymers, such as, for example, hydroxyethylcellulose, starch or acrylates.

Oxidation dyes which can be used are coupler substances and developer substances, and self-coupling color precursors.

The oxidation color precursors are used in the dye carrier mass either in the form of the free base or in the form of their physiologically acceptable salts with inorganic or organic acids, for example as chloride, sulfate, phosphate, acetate, propionate, lactate or citrate, or—if they have aromatic OH groups—in the form of the salts with bases, for example as alkali metal phenoxides.

Suitable developer substances are preferably 1,4-diaminobenzene (p-phenylenediamine), 1,4-diamino-2-methylbenzene (p-tolylenediamine), 1,4-diamino-2,6-dimethylbenzene, 1,4-diamino-3,5-diethylbenzene, 1,4-diamino-2,5-dimethylbenzene, 1,4-diamino-2,3-dimethylbenzene, 2-chloro-1,4-diaminobenzene, 1,4-diamino-2-(thiophen-2-yl)benzene, 1,4-diamino-2-(thiophen-3-yl)benzene, 4-(2,5-diaminophenyl)-2-((diethylamino)methyl)thiophene, 2-chloro-3-(2,5-diaminophenyl)thiophene, 1,4-diamino-2-(pyridin-3-yl)benzene, 2,5-diaminobiphenyl, 2,5-diamino-4′-(1-methylethyl)-1,1′-biphenyl, 2,3′,5-triamino-1,1′-biphenyl, 1,4-diamino-2-methoxymethylbenzene, 1,4-diamino-2-aminomethylbenzene, 1,4-diamino-2-((phenylamino)methyl)benzene, 1,4-di-amino-2-((ethyl-(2-hydroxyethyl)amino)methyl)benzene, 1,4-diamino-2-hydroxymethylbenzene, 1,4-diamino-2-(2-hydroxyethoxy)benzene, 2-(2-(acetylamino)ethoxy)-1,4-diamino-benzene, 4-phenylaminoaniline, 4-dimethylamino-aniline, 4-diethylaminoaniline, 4-dipropylaminoaniline, 4-[ethyl(2-hydroxyethyl)amino]aniline, 4-[di(2-hydroxyethyl)amino]aniline, 4-[di(2-hydroxyethyl)amino]-2-methylaniline, 4-[(2-methoxyethyl)amino]aniline, 4-[(3-hydroxypropyl)amino]aniline, 4-[(2,3-dihydroxypropyl)amino]aniline, 4-(((4-aminophenyl)methyl)amino)aniline, 4-[(4-aminophenylamino)methyl]phenol, 1,4-diamino-N-(4-pyrrolidin-1-ylbenzyl)benzene, 1,3-dihydroxy-2-((2-furylmethyl)aminomethyl)benzene, 1,4-diamino-N-thiophen-2-ylmethylbenzene, 1,4-diamino-N-furan-2-ylmethylbenzene, 1,4-diamino-N-thiophen-3-ylmethylbenzene, 1,4-diamino-N-benzylbenzene, 1,4-diamino-2-(1-hydroxyethyl)benzene, 1,4-diamino-2-(2-hydroxyethyl)benzene, 1,4-diamino-2-(1-methylethyl)benzene, 1,3-bis[(4-amino-phenyl)(2-hydroxyethyl)amino]-2-propanol, 1,4-bis[(4-aminophenyl)amino]butane, 1,8-bis(2,5-diaminophenoxy)-3,6-dioxaoctane, 2,5-diamino-4′-hydroxy-1,1′-biphenyl, 2,5-diamino-2′-trifluoromethyl-1,1′-biphenyl, 2,4′,5-triamino-1,1′-biphenyl, 4-aminophenol, 4-amino-3-methylphenol, 4-amino-3-(hydroxymethyl)phenol, 4-amino-3-fluorophenol, 4-methylamino-phenol, 4-amino-2-(aminomethyl)phenol, 4-amino-2-(hydroxymethyl)phenol, 4-amino-2-fluorophenol, 4-amino-2-[(2-hydroxyethyl)amino]-methylphenol, 4-amino-2-methylphenol, 4-amino-2-(methoxymethyl)phenol, 4-amino-2-(2-hydroxyethyl)phenol, 5-aminosalicylic acid, 2,5-diaminopyridine, 2,4,5,6-tetraaminopyrimidine, 2,5,6-triamino-4-(1H)-pyrimidone, 4,5-diamino-1-(2-hydroxyethyl)-1H-pyrazole, 4,5-diamino-1-(1-methylethyl)-1H-pyrazole, 4,5-diamino-1-[(4-methylphenyl)methyl]-1H-pyrazole, 1-[(4-chlorophenyl)methyl]-4,5-diamino-1H-pyrazole, 4,5-diamino-1-methyl-1H-pyrazole, 4,5-diamino-1-pentyl-1H-pyrazole, 4,5-diamino-1-(phenylmethyl)-1H-pyrazole, 4,5-diamino-1-((4-methoxyphenyl)methyl-1H-pyrazole, 2-aminophenol, 2-amino-6-methylphenol, 2-amino-5-methylphenol, 1,2,4-trihydroxybenzene, 2,4-diaminophenol, 1,4-dihydroxybenzene, 2-(((4-amino-phenyl)amino)methyl)-1,4-diaminobenzene; where 1,4-diaminobenzene, 1,4-diamino-2-methylbenzene, 2-(2′-hydroxyethyl)-1,4-diaminobenzene, 1,4-diamino-2-hydroxymethylbenzene, 1,4-diamino-2-methoxymethylbenzene, 2,5-diaminobiphenyl, 4-aminophenol, 4-methylaminophenol, 4-amino-3-methylphenol and 4-bis(2′-hydroxyethyl)aminoaniline and 4,5-diamino-1-(2-hydroxyethyl)-1H-pyrazole are preferred, and 4,5-diamino-1-(2-hydroxyethyl)-1H-pyrazole, 2,5-diamino-biphenyl, 1,4-diamino-2-methoxymethylbenzene and 2-(2′-hydroxyethyl)-1,4-diaminobenzene are particularly preferred.

Examples of coupler substances are: N-(3-dimethylamino-phenyl)urea, 2,6-diaminopyridine, 2-amino-4-[(2-hydroxyethyl)amino]anisole, 2,4-diamino-1-fluoro-5-methylbenzene, 2,4-diamino-1-methoxy-5-methylbenzene, 2,4-diamino-1-ethoxy-5-methylbenzene, 2,4-diamino-1-(2-hydroxyethoxy)-5-methylbenzene, 2,4-di[(2-hydroxy-ethyl)amino]-1,5-dimethoxybenzene, 2,3-diamino-6-methoxypyridine, 3-amino-6-methoxy-2-(methylamino)pyridine, 2,6-diamino-3,5-dimethoxypyridine, 3,5-diamino-2,6-dimethoxypyridine, 1,3-diaminobenzene, 2,4-diamino-1-(2-hydroxyethoxy)benzene, 1,3-diamino-4-(2,3-dihydroxypropoxy)benzene, 1,3-diamino-4-(3-hydroxypropoxy)benzene, 1,3-diamino-4-(2-methoxyethoxy)benzene, 2,4-diamino-1,5-di(2-hydroxy-ethoxy)benzene, 1-(2-aminoethoxy)-2,4-diaminobenzene, 2-amino-1-(2-hydroxyethoxy)-4-methylaminobenzene, 2,4-diaminophenoxyacetic acid, 3-[di(2-hydroxyethyl)amino]aniline, 4-amino-2-di[(2-hydroxyethyl)amino]-1-ethoxybenzene, 5-methyl-2-(1-methylethyl)phenol, 3-[(2-hydroxyethyl)amino]aniline, 3-[(2-aminoethyl)amino]aniline, 1,3-di(2,4-diaminophenoxy)propane, di(2,4-diaminophenoxy)-methane, 1,3-diamino-2,4-dimethoxybenzene, 2,6-bis(2-hydroxyethyl)aminotoluene, 4-hydroxyindole, 3-dimethylaminophenol, 3-diethylaminophenol, 5-amino-2-methylphenol, 5-amino-4-fluoro-2-methylphenol, 5-amino-4-methoxy-2-methylphenol, 5-amino-4-ethoxy-2-methylphenol, 3-amino-2,4-dichlorophenol, 5-amino-2,4-dichlorophenol, 3-amino-2-methylphenol, 3-amino-2-chloro-6-methylphenol, 3-aminophenol, 2-[(3-hydroxyphenyl)amino]acetamide, 5-[(2-hydroxyethyl)amino]-4-methoxy-2-methylphenol, 5-[(2-hydroxyethyl)amino]-2-methylphenol, 3-[(2-hydroxyethyl)amino]phenol, 3-[(2-methoxyethyl)amino]phenol, 5-amino-2-ethylphenol, 5-amino-2-methoxyphenol, 2-(4-amino-2-hydroxyphenoxy)ethanol, 5-[(3-hydroxypropyl)amino]-2-methylphenol, 3-[(2,3-dihydroxypropyl)amino]-2-methylphenol, 3-[(2-hydroxyethyl)amino]-2-methylphenol, 2-amino-3-hydroxypyridine, 2,6-dihydroxy-3,4-dimethylpyridine, 5-amino-4-chloro-2-methylphenol, 1-naphthol, 2-methyl-1-naphthol, 1,5-dihydroxynaphthalene, 1,7-dihydroxynaphthalene, 2,3-dihydroxynaphthalene, 2,7-dihydroxy-naphthalene, 2-methyl-1-naphthol acetate, 1,3-di-hydroxybenzene, 1-chloro-2,4-dihydroxybenzene, 2-chloro-1,3-dihydroxybenzene, 1,2-dichloro-3,5-dihydroxy-4-methylbenzene, 1,5-dichloro-2,4-dihydroxybenzene, 1,3-dihydroxy-2-methylbenzene, 3,4-methylenedioxyphenol, 3,4-methylenedioxyaniline, 5-[(2-hydroxyethyl)amino]-1,3-benzodioxole, 6-bromo-1-hydroxy-3,4-methylenedioxybenzene, 3,4-diaminobenzoic acid, 3,4-dihydro-6-hydroxy-1,4(2H)-benzoxazine, 6-amino-3,4-dihydro-1,4(2H)-benzoxazine, 3-methyl-1-phenyl-5-pyrazolone, 5,6-dihydroxyindole, 5,6-dihydroxyindoline, 5-hydroxyindole, 6-hydroxyindole, 7-hydroxyindole and 2,3-indolinedione; where 1,3-dihydroxybenzene, 1-chloro-2,4-dihydroxybenzene, 1,3-dihydroxy-2-methyl-benzene, 3,4-methylenedioxyphenol, 2,6-diaminopyridine, 2,4-diamino-1-(2-hydroxyethoxy)benzene, 2-amino-4-[(2-hydroxyethyl)amino]anisole, 3-aminophenol, 5-amino-2-methylphenol, 5-[(2-hydroxyethyl)amino]-2-methylphenol, 1-naphthol, 2-methyl-1-naphthol, 5-amino-4-fluoro-2-methylphenol, 1,5-dihydroxynaphthalene, 1,7-dihydroxynaphthalene, 4-hydroxyindole, 3,4-methylenedioxyaniline, N-(2′-hydroxyethyl)-3,4-methylenedioxyaniline and 3-methyl-1-phenyl-5-pyrazolone, and in particular 5-amino-2-methylphenol, 1-naphthol, 2,4-diamino-1-(2-hydroxyethoxy)benzene, 5-[(2-hydroxyethyl)amino]-2-methylphenol and N-(2′-hydroxyethyl)-3,4-methylene-dioxyaniline are particularly preferred.

The coupler substances and developer substances are preferably used in approximately equimolar amounts. However, it is not disadvantageous if the coupler substances are used in an excess or shortfall compared with the developer substances, it being possible for both the developer component to be a mixture of known developer substances, and also for the coupler component to be a mixture of known coupler substances.

Self-coupling color precursors are, for example: 2-amino-6-methylphenol, 2-amino-5-methylphenol and 2-amino-5-phenylphenol.

The total amount of the oxidation color precursors present in the dye carrier mass is about 0.01 to 12 percent by weight, particular preference being given to an amount of from about 0.02 to 10 percent by weight and especially 0.2 to 6 percent by weight.

In addition, to achieve certain color nuances customary natural or synthetic direct dyes from the group of acidic and basic dyes, triphenylmethane dyes, aromatic nitro dyes, azo dyes and anthraquinone dyes may also be present in the dye carrier mass. The total amount of the direct dyes in the dye carrier mass according to the invention is 0.01 to 7 percent by weight, preferably 0.2 to 3.0 percent by weight.

The nonionic organic thickeners with wax-like properties used are preferably one or more C12- to C24-fatty alcohols, in particular cetyl alcohol and/or stearyl alcohol, and glyceryl monooleate and mixtures of glyceryl monooleate and one or more C12- to C24-fatty alcohols, in particular mixtures of glyceryl monooleate and cetyl alcohol and/or stearyl alcohol.

The amount of nonionic organic thickeners with wax-like properties used is about 5 to 25 percent by weight, preferably 10 to 20 percent by weight.

Although the specified thickeners are entirely adequate for fulfilling the set object, further wax components, such as, for example, diglyceryl diisostearates, stearic acid monoethanolamide, cocamide monoethanolamide, Vaseline, glycerol esters of saturated or unsaturated fatty acids, glycol disearates, glycol stearates, cetyl palmitates, beeswax and other cosmetically customary wax components can additionally be co-used.

The nonionic surfactant used is a mixture of at least one sorbitan fatty acid ester oxyethylated with 4 to 20 mol of ethylene oxide and at least one C12-C24-fatty alcohol oxyethylated with 30 to 70 mol of ethylene oxide, preference being given to a mixture of sorbitan lauric esters, sorbitan palmitic esters or sorbitan stearic esters ethoxylated with 20 mol of ethylene oxide (polysorbate 20, polysorbate 40, polysorbate 60) and cetyl alcohol, stearyl alcohol or cetylstearyl alcohol ethoxylated with 30 to 70 mol of ethylene oxide (ceteth-30 to 70, steareth-30 to 70, ceteareth-30 to 70), and in particular to a mixture of a sorbitan palmitic ester ethoxylated with 20 mol of ethylene oxide (polysorbate 40) and a cetylstearyl alcohol ethoxylated with 50 mol of ethylene oxide (ceteareth-50).

The amount of nonionogenic surfactants used is about 3 to 25 percent by weight, preferably 5 to 20 percent by weight.

Besides the nonionic organic thickeners with wax-like properties and the nonionic surfactants, the dye carrier mass comprises customary cosmetic cationic resins, particular preference being given to poly(dimethyldiallylammonium chloride) (=polyquaternium-6), diethyldiallylammonium chloride/acrylamide copolymer (=polyquaternium-7), cationic cellulose (=polyquaternium-10), N,N-dimethylaminoethylmethacrylic acid/PVP copolymer diethyl sulfate (=polyquaternium-11) and polyquaternium-35, alone or in a mixture with one another. The total amount of these cationic resins in the dye carrier mass is about 0.1 to 6 percent by weight.

The dye carrier mass according to the invention can optionally comprise special care substances and/or active ingredients, such as, for example, amino acids, protein hydrolysates, for example keratin hydrolysate, elastin hydrolysates, collagen hydrolysates, silk protein hydrolysates, milk protein hydrolysates, soya protein hydrolysates or wheat protein hydrolysates, panthenol, allantoin, creatine/creatinine, p-aminobenzoic acid, linolenic acid or salts thereof, pyrrolidonecarboxylic acid or salts thereof, plant extracts or vitamins—alone or in combination with one another, these compounds being present in the color carrier mass preferably in a total concentration of from about 0.01 to 5 percent by weight, in particular 0.01 to 1 percent by weight.

Moreover, the color carrier mass can comprise care oils of vegetable origin, for example wheat germ oil, sunflower oil, jojoba oil, or of mineral origin, for example paraffin oil, isopropyl myristate, oleth-2. Additionally, antioxidants such as ascorbic acid, ascorbyl palmitate, thioglycolic acid or sodium sulfite, complexing agents for heavy metals, such as EDTA or nitrilotriacetic acid, and perfume oils can be added.

Depending on the composition, the dye carrier mass according to the invention can have a weakly acidic, neutral or alkaline reaction; in particular it has a pH of from about 6 to 13, basic adjustment preferably taking place with ammonia. However, it is also possible to use organic amines, for example monoethanolamine or tris(hydroxymethyl)aminomethane, or inorganic bases, such as sodium hydroxide or potassium hydroxide. When using organic amines, these can be used on their own or in a mixture with ammonia.

For a pH range in the acidic range, inorganic or organic acids, for example phosphoric acid, citric acid or tartaric acid, are suitable.

Moreover, the novel color carrier mass can comprise customary monohydric and polyhydric alcohols, such as, for example, ethanol, isopropyl alcohol, 1,3-butanediol, glycerol or 1,2-propylene glycol.

For the use for the oxidative coloring of hair, the color carrier mass described above is mixed directly prior to use with a liquid or emulsion-like oxidizing agent and an amount of this mixture sufficient for the hair-coloring treatment, generally about 50 to 200 grams, depending on the fullness of the hair, is applied to the hair.

The ready-to-use oxidation hair colorant obtained after mixing the dye carrier mass with the oxidizing agent preferably has a pH of from about 6.5 to 12.

Suitable oxidizing agents for developing the hair coloration are primarily hydrogen peroxide or its addition compounds onto urea, melamine, sodium borate or sodium carbonate in the form of a 3 to 12 percent strength, preferably 6 percent strength, aqueous solution, but also atmospheric oxygen. If a 6 percent strength hydrogen peroxide solution is used as oxidizing agent, then the weight ratio between hair colorant and oxidizing agent is 5:1 to 1:4, but preferably 2:1 to 1:3. Relatively large amounts of oxidizing agent are used primarily for relatively high dye concentrations in the hair colorant, or if stronger bleaching of the hair is intended at the same time.

The mixture is left to act on the hair at 15 to 50 degrees Celsius for about 10 to 45 minutes, preferably 15 to 30 minutes, then the hair is rinsed with water and dried. If required, this rinsing is followed by washing with a shampoo and possibly after-rinsing with a weak organic acid, such as, for example, citric acid or tartaric acid. The hair is then dried.

The present invention further provides a two-component composition for the oxidative coloring of hair consisting of two components A and B to be mixed directly prior to application, which is characterized in that the above-described dye carrier mass is used as component A, and an aqueous liquid or cream-like oxidizing agent preparation with a content of about 1 to 12 percent by weight of an oxidizing agent, preferably hydrogen peroxide, is used as component B. If a cream-like preparation is used, this additionally comprises about 1 to 8 percent by weight of a C12- to C24-fatty alcohol of natural or synthetic origin.

Compared with standard commercial compositions, the hair colorant according to the invention has a considerably improved hair-conditioning effect and improved skin compatibility. The improved hair care effect is also more resistant to shampoo, i.e. it withstands more hair washes.

The examples below are intended to illustrate the subject-matter of the invention in more detail without limiting this to the examples.

### EXAMPLES

In the examples below the following abbreviations are used for the dyes used:
A: 1,4-diamino-2-methylbenzene hydrosulfate
B: 4-bis(2′-hydroxyethyl)aminoaniline
C: 4,5-diamino-1-(2-hydroxyethyl)-1H-pyrazole hydrosulfate
D: 2-(2′-hydroxyethyl)-1,4-diaminobenzene sulfate
E: 4-aminophenol
F: 4-amino-3-methylphenol
G: 2,5-diaminobiphenyl
H: 1,4-diamino-2-methoxymethylbenzene
I: 1,3-dihydroxybenzene
J: 3-aminophenol
K: 5-amino-2-methylphenol
L: 1,3-diamino-4-(2′-hydroxyethoxy)benzene hydrochloride
M: 1,3-dihydroxy-2-methylbenzene
N: 1-naphthol
O: N-(2′-hydroxyethyl)-3,4-methylenedioxyaniline hydrochloride
P: 5-[(2-hydroxyethyl)amino]-2-methylphenol
Q: 2-amino-4-(2′-hydroxyethyl)aminoanisole
R: 2-amino-6-chloro-4-nitrophenol
S: N-(2′-hydroxyethyl)-2-nitro-4-(trifluoromethyl)aniline
T: 2-amino-4,6-dinitrophenol
U: 2-chloro-6-ethylamino-4-nitrophenol

### Examples 1 to 126

### Oxidation Hair Colorants, Cream-Like

Dye carrier mass

| | | |
|---|---|---|
| 8.0 | g | cetearyl alcohol |
| 3.0 | g | glyceryl oleate |
| 13.0 | g | polysorbate-40 |
| 5.0 | g | ceteareth-50 |
| 1.2 | g | polyquaternium-6 |
| 0.4 | g | sodium sulfite, anhydrous |
| 0.5 | g | sodium sulfate, anhydrous |
| 0.2 | g | ascorbic acid |
| 0.1 | g | 1-hydroxyethane-1,1-diphosphonic acid |
| X | g | dye combination according to Table 1 |
| 1.5 | g | monoethanolamine, 85% strength aqueous solution |
| 5.8 | g | ammonia, 25% strength aqueous solution |
| 0.3 | g | perfume |
| ad 100.0 | g | water |

Hydrogen peroxide emulsion (pH = 2.5)

| | | |
|---|---|---|
| 3.0 | g | cetylstearyl alcohol |
| 0.8 | g | PEG-25 cetyl/stearyl ether |
| 0.3 | g | lanolin alcohol |
| 6.0 | g | hydrogen peroxide |
| 0.1 | g | phosphoric acid, 85% strength aqueous solution |
| ad 100.0 | g | water |

20 g of the above coloring cream are mixed directly prior to application with 20 g of the 6% strength hydrogen peroxide emulsion. The mixture obtained in this way (pH=9.5-10.5) is applied to bleached hair tresses and, after a contact time of 30 minutes at room temperature, rinsed out with water. The hair is then washed with a standard commercial shampoo and then dried. The dye combinations used and also the resulting coloring ring results are summarized in Table 1 below.

TABLE 1

| Ex. | | |
|---|---|---|
| No. | Dye combination | Color |
| 1 | 2.5 mmol A + 0.875 mmol I + 0.875 mmol J + 0.5 mmol K + 0.25 mmol L | gray-pink, |
| | | medium |
| 2 | 2.1 mmol A + 0.73 mmol I + 0.73 mmol J + 0.42 mmol K + 0.21 mmol | brown-gray, |
| | L + 0.42 mmol R + 0.42 mmol S | dark |
| 3 | 2.1 mmol A + 0.73 mmol I + 0.73 mmol J + 0.42 mmol K + 0.21 mmol | brown-gray, |
| | L + 0.42 mmol T + 0.42 mmol U | medium |
| 4 | 2.5 mmol B + 0.875 mmol I + 0.875 mmol J + 0.5 mmol K + 0.25 mmol L | blue |
| 5 | 2.1 mmol B + 0.73 mmol I + 0.73 mmol J + 0.42 mmol K + 0.21 mmol | blue-green |
| | L + 0.42 mmol R + 0.42 mmol S | |
| 6 | 2.1 mmol B + 0.73 mmol I + 0.73 mmol J + 0.42 mmol K + 0.21 mmol | gray |
| | L + 0.42 mmol T + 0.42 mmol U | |
| 7 | 2.5 mmol C + 0.875 mmol I + 0.875 mmol J + 0.5 mmol K + 0.25 mmol L | red, medium |
| 8 | 2.1 mmol C + 0.73 mmol I + 0.73 mmol J + 0.42 mmol K + 0.21 mmol | red-orange, |
| | L + 0.42 mmol R + 0.42 mmol S | pale |
| 9 | 2.1 mmol C + 0.73 mmol I + 0.73 mmol J + 0.42 mmol K + 0.21 mmol | red-yellow, |
| | L + 0.42 mmol T + 0.42 mmol U | medium |
| 10 | 2.5 mmol D + 0.875 mmol I + 0.875 mmol J + 0.5 mmol K + 0.25 mmol L | gray-pink, |
| | | pale |
| 11 | 2.1 mmol D + 0.73 mmol I + 0.73 mmol J + 0.42 mmol K + 0.21 mmol | gray-yellow, |
| | L + 0.42 mmol R + 0.42 mmol S | medium |
| 12 | 2.1 mmol D + 0.73 mmol I + 0.73 mmol J + 0.42 mmol K + 0.21 mmol | red-gray |
| | L + 0.42 mmol T + 0.42 mmol U | |
| 13 | 2.5 mmol E + 0.875 mmol I + 0.875 mmol J + 0.5 mmol K + 0.25 mmol L | pink-orange, |
| | | pale |
| 14 | 2.1 mmol E + 0.73 mmol I + 0.73 mmol J + 0.42 mmol K + 0.21 mmol | orange, pale |
| | L + 0.42 mmol R + 0.42 mmol S | |
| 15 | 2.1 mmol E + 0.73 mmol I + 0.73 mmol J + 0.42 mmol K + 0.21 mmol | blue-orange, |
| | L + 0.42 mmol T + 0.42 mmol U | pale |
| 16 | 2.5 mmol F + 0.875 mmol I + 0.875 mmol J + 0.5 mmol K + 0.25 mmol L | pale blonde |
| | | pink |
| 17 | 2.1 mmol F + 0.73 mmol I + 0.73 mmol J + 0.42 mmol K + 0.21 mmol | matt orange, |
| | L + 0.42 mmol R + 0.42 mmol S | pale |
| 18 | 2.1 mmol F + 0.73 mmol I + 0.73 mmol J + 0.42 mmol K + 0.21 mmol | orange-pink, |
| | L + 0.42 mmol T + 0.42 mmol U | pale |
| 19 | 2.5 mmol G + 0.875 mmol I + 0.875 mmol J + 0.5 mmol K + 0.25 mmol L | pale gray |
| 20 | 2.1 mmol G + 0.73 mmol I + 0.73 mmol J + 0.42 mmol K + 0.21 mmol | brown-gray, |
| | L + 0.42 mmol R + 0.42 mmol S | pale |
| 21 | 2.1 mmol F + 0.73 mmol I + 0.73 mmol J + 0.42 mmol K + 0.21 mmol | gray-pink, |
| | L + 0.42 mmol T + 0.42 mmol U | pale |
| 22 | 2.5 mmol H + 0.875 mmol I + 0.875 mmol J + 0.5 mmol K + 0.25 mmol L | gray-yellow, |
| | | dark |
| 23 | 2.1 mmol H + 0.73 mmol I + 0.73 mmol J + 0.42 mmol K + 0.21 mmol | gray-pink |
| | L + 0.42 mmol R + 0.42 mmol S | |
| 24 | 2.1 mmol H + 0.73 mmol I + 0.73 mmol J + 0.42 mmol K + 0.21 mmol | pink-gray |
| | L + 0.42 mmol T + 0.42 mmol U | |
| 25 | 2.5 mmol A + 0.75 mmol M + 0.5 mmol N + 0.5 mmol O + 0.5 mmol | violet-pink, |
| | P + 0.25 mmol Q | dark |
| 26 | 2.1 mmol A + 0.63 mmol M + 0.42 mmol N + 0.42 mmol O + 0.42 mmol | violet-brown |
| | P + 0.21 mmol Q + 0.42 mmol R + 0.42 mmol S | pink |
| 27 | 2.1 mmol A + 0.63 mmol M + 0.42 mmol N + 0.42 mmol O + 0.42 mmol | violet-brown |
| | P + 0.21 mmol Q + 0.42 mmol T + 0.42 mmol U | pink, pale |
| 28 | 2.5 mmol B + 0.75 mmol M + 0.5 mmol N + 0.5 mmol O + 0.5 mmol | violet |
| | P + 0.25 mmol Q | |
| 29 | 2.1 mmol B + 0.63 mmol M + 0.42 mmol N + 0.42 mmol O + 0.42 mmol | gray-violet |
| | P + 0.21 mmol Q + 0.42 mmol R + 0.42 mmol S | |
| 30 | 22.1 mmol B + 0.63 mmol M + 0.42 mmol N + 0.42 mmol O + 0.42 mmol | gray-pink, |
| | P + 0.21 mmol Q + 0.42 mmol T + 0.42 mmol U | dark |
| 31 | 2.5 mmol C + 0.75 mmol M + 0.5 mmol N + 0.5 mmol | red-blue, |
| | O + 0.5 mmol P + 0.25 mmol Q | medium |
| 32 | 2.1 mmol C + 0.63 mmol M + 0.42 mmol N + 0.42 mmol O + 0.42 mmol | red-orange, |
| | P + 0.21 mmol Q + 0.42 mmol R + 0.42 mmol S | dark |
| 33 | 2.1 mmol C + 0.63 mmol M + 0.42 mmol N + 0.42 mmol O + 0.42 mmol | orange-blue, |
| | P + 0.21 mmol Q + 0.42 mmol T + 0.42 mmol U | dark |
| 34 | 2.5 mmol D + 0.75 mmol M + 0.5 mmol N + 0.5 mmol | pink-violet |
| | O + 0.5 mmol P + 0.25 mmol Q | gray |
| 35 | 2.1 mmol D + 0.63 mmol M + 0.42 mmol N + 0.42 mmol O + 0.42 mmol | pink-violet |
| | P + 0.21 mmol Q + 0.42 mmol R + 0.42 mmol S | matt, pale |
| 36 | 2.1 mmol D + 0.63 mmol M + 0.42 mmol N + 0.42 mmol O + 0.42 mmol | red-violet, |
| | P + 0.21 mmol Q + 0.42 mmol T + 0.42 mmol U | pale |
| 37 | 2.5 mmol E + 0.75 mmol M + 0.5 mmol N + 0.5 mmol | pink-orange |
| | O + 0.5 mmol P + 0.25 mmol Q | |
| 38 | 2.1 mmol E + 0.63 mmol M + 0.42 mmol N + 0.42 mmol O + 0.42 mmol | orange |
| | P + 0.21 mmol Q + 0.42 mmol R + 0.42 mmol S | |
| 39 | 2.1 mmol E + 0.63 mmol M + 0.42 mmol N + 0.42 mmol O + 0.42 mmol | blue-orange |
| | P + 0.21 mmol Q + 0.42 mmol T + 0.42 mmol U | |
| 40 | 2.5 mmol F + 0.75 mmol M + 0.5 mmol N + 0.5 mmol | medium |
| | O + 0.5 mmol P + 0.25 mmol Q | blonde pink |
| 41 | 2.1 mmol F + 0.63 mmol M + 0.42 mmol N + 0.42 mmol O + 0.42 mmol | matt orange |
| | P + 0.21 mmol Q + 0.42 mmol R + 0.42 mmol S | |
| 42 | 2.1 mmol F + 0.63 mmol M + 0.42 mmol N + 0.42 mmol O + 0.42 mmol | orange-pink |
| | P + 0.21 mmol Q + 0.42 mmol T + 0.42 mmol U | |
| 43 | 2.5 mmol G + 0.75 mmol M + 0.5 mmol N + 0.5 mmol | violet-gray |
| | O + 0.5 mmol P + 0.25 mmol Q | |
| 44 | 2.1 mmol G + 0.63 mmol M + 0.42 mmol N + 0.42 mmol O + 0.42 mmol | brown-gray |
| | P + 0.21 mmol Q + 0.42 mmol R + 0.42 mmol S | |
| 45 | 2.1 mmol G + 0.63 mmol M + 0.42 mmol N + 0.42 mmol O + 0.42 mmol | brown-pink |
| | P + 0.21 mmol Q + 0.42 mmol T + 0.42 mmol U | gray |
| 46 | 2.5 mmol H + 0.75 mmol M + 0.5 mmol N + 0.5 mmol | pink-violet |
| | O + 0.5 mmol P + 0.25 mmol Q | gray |
| 47 | 2.1 mmol H + 0.63 mmol M + 0.42 mmol N + 0.42 mmol O + 0.42 mmol | violet-brown |
| | P + 0.21 mmol Q + 0.42 mmol R + 0.42 mmol S | gray |
| 48 | 2.1 mmol H + 0.63 mmol M + 0.42 mmol N + 0.42 mmol O + 0.42 mmol | pink-brown |
| | P + 0.21 mmol Q + 0.42 mmol T + 0.42 mmol U | |
| 49 | 1.25 mmol A + 1.25 mmol E + 0.875 mmol I + 0.875 mmol J + 0.5 mmol | violet-beige |
| | K + 0.25 mmol L | |
| 50 | 1.04 mmol A + 1.04 mmol E + 0.73 mmol I + 0.73 mmol J + 0.42 mmol | gold-beige |
| | K + 0.21 mmol L + 0.42 mmol R + 0.42 mmol S | |
| 51 | 1.04 mmol A + 1.04 mmol E + 0.73 mmol I + 0.73 mmol J + 0.42 mmol | orange-beige |
| | K + 0.21 mmol L + 0.42 mmol T + 0.42 mmol U | |
| 52 | 1.25 mmol B + 1.25 mmol E + 0.875 mmol I + 0.875 mmol J + 0.5 mmol | gray |
| | K + 0.25 mmol L | |
| 53 | 1.04 mmol B + 1.04 mmol E + 0.73 mmol I + 0.73 mmol J + 0.42 mmol | matt brown |
| | K + 0.21 mmol L + 0.42 mmol R + 0.42 mmol S | |
| 54 | 1.04 mmol B + 1.04 mmol E + 0.73 mmol I + 0.73 mmol J + 0.42 mmol | orange- |
| | K + 0.21 mmol L + 0.42 mmol T + 0.42 mmol U | brown matt |
| 55 | 1.25 mmol C + 1.25 mmol E + 0.875 mmol I + 0.875 mmol J + 0.5 mmol | red, pale |
| | K + 0.25 mmol L | |
| 56 | 1.04 mmol C + 1.04 mmol E + 0.73 mmol I + 0.73 mmol J + 0.42 mmol | red-orange, |
| | K + 0.21 mmol L + 0.42 mmol R + 0.42 mmol S | dark |
| 57 | 1.04 mmol C + 1.04 mmol E + 0.73 mmol I + 0.73 mmol J + 0.42 mmol | red-yellow, |
| | K + 0.21 mmol L + 0.42 mmol T + 0.42 mmol U | dark |
| 58 | 1.25 mmol D + 1.25 mmol E + 0.875 mmol I + 0.875 mmol J + 0.5 mmol | beige-violet |
| | K + 0.25 mmol L | matt |
| 59 | 1.04 mmol D + 1.04 mmol E + 0.73 mmol I + 0.73 mmol J + 0.42 mmol | gold-beige |
| | K + 0.21 mmol L + 0.42 mmol R + 0.42 mmol S | |
| 60 | 1.04 mmol D + 1.04 mmol E + 0.73 mmol I + 0.73 mmol J + 0.42 mmol | beige-orange |
| | K + 0.21 mmol L + 0.42 mmol T + 0.42 mmol U | |
| 61 | 1.25 mmol E + 1.25 mmol F + 0.875 mmol I + 0.875 mmol J + 0.5 mmol | medium |
| | K + 0.25 mmol L | blonde pink |
| 62 | 1.04 mmol E + 1.04 mmol F + 0.73 mmol I + 0.73 mmol J + 0.42 mmol | beige- |
| | K + 0.21 mmol L + 0.42 mmol R + 0.42 mmol S | orange, pale |
| 63 | 1.04 mmol E + 1.04 mmol F + 0.73 mmol I + 0.73 mmol J + 0.42 mmol | red-orange |
| | K + 0.21 mmol L + 0.42 mmol T + 0.42 mmol U | |
| 64 | 1.25 mmol E + 1.25 mmol G + 0.875 mmol I + 0.875 mmol J + 0.5 mmol | pale brown |
| | K + 0.25 mmol L | pink |
| 65 | 1.04 mmol E + 1.04 mmol G + 0.73 mmol I + 0.73 mmol J + 0.42 mmol | beige-blonde |
| | K + 0.21 mmol L + 0.42 mmol R + 0.42 mmol S | |
| 66 | 1.04 mmol E + 1.04 mmol G + 0.73 mmol I + 0.73 mmol J + 0.42 mmol | beige- |
| | K + 0.21 mmol L + 0.42 mmol T + 0.42 mmol U | orange, dark |
| 67 | 1.25 mmol E + 1.25 mmol H + 0.875 mmol I + 0.875 mmol J + 0.5 mmol | matt pink, |
| | K + 0.25 mmol L | medium |
| 68 | 1.04 mmol E + 1.04 mmol H + 0.73 mmol I + 0.73 mmol J + 0.42 mmol | beige-orange |
| | K + 0.21 mmol L + 0.42 mmol R + 0.42 mmol S | |
| 69 | 1.04 mmol E + 1.04 mmol H + 0.73 mmol I + 0.73 mmol J + 0.42 mmol | beige- |
| | K + 0.21 mmol L + 0.42 mmol T + 0.42 mmol U | orange, dark |
| 70 | 1.25 mmol A + 1.25 mmol E + 0.75 mmol M + 0.5 mmol N + 0.5 mmol | dark brown |
| | O + 0.5 mmol P + 0.25 mmol Q | pink |
| 71 | 1.04 mmol A + 1.04 mmol E + 0.73 mmol I + 0.73 mmol J + 0.42 mmol | pale brown |
| | K + 0.21 mmol L + 0.42 mmol R + 0.42 mmol S | pink |
| 72 | 1.04 mmol A + 1.04 mmol E + 0.73 mmol I + 0.73 mmol J + 0.42 mmol | red-brown |
| | K + 0.21 mmol L + 0.42 mmol T + 0.42 mmol U | |
| 73 | 1.25 mmol B + 1.25 mmol E + 0.75 mmol M + 0.5 mmol N + 0.5 mmol | violet-pale |
| | O + 0.5 mmol P + 0.25 mmol Q | brown |
| 74 | 1.04 mmol B + 1.04 mmol E + 0.73 mmol I + 0.73 mmol J + 0.42 mmol | dark beige |
| | K + 0.21 mmol L + 0.42 mmol R + 0.42 mmol S | |
| 75 | 1.04 mmol B + 1.04 mmol E + 0.73 mmol I + 0.73 mmol J + 0.42 mmol | beige- |
| | K + 0.21 mmol L + 0.42 mmol T + 0.42 mmol U | orange, dark |
| 76 | 1.25 mmol C + 1.25 mmol E + 0.75 mmol M + 0.5 mmol N + 0.5 mmol | red-blue, |
| | O + 0.5 mmol P + 0.25 mmol Q | dark |
| 77 | 1.04 mmol C + 1.04 mmol E + 0.73 mmol I + 0.73 mmol J + 0.42 mmol | red-orange, |
| | K + 0.21 mmol L + 0.42 mmol R + 0.42 mmol S | medium |
| 78 | 1.04 mmol C + 1.04 mmol E + 0.73 mmol I + 0.73 mmol J + 0.42 mmol | orange-blue, |
| | K + 0.21 mmol L + 0.42 mmol T + 0.42 mmol U | medium |
| 79 | 1.25 mmol D + 1.25 mmol E + 0.75 mmol M + 0.5 mmol N + 0.5 mmol | matt pink, |
| | O + 0.5 mmol P + 0.25 mmol Q | dark |
| 80 | 1.04 mmol D + 1.04 mmol E + 0.73 mmol I + 0.73 mmol J + 0.42 mmol | blonde |
| | K + 0.21 mmol L + 0.42 mmol R + 0.42 mmol S | orange |
| 81 | 1.04 mmol D + 1.04 mmol E + 0.73 mmol I + 0.73 mmol J + 0.42 mmol | blue-orange |
| | K + 0.21 mmol L + 0.42 mmol T + 0.42 mmol U | |
| 82 | 1.25 mmol E + 1.25 mmol F + 0.75 mmol M + 0.5 mmol N + 0.5 mmol | dark blonde |
| | O + 0.5 mmol P + 0.25 mmol Q | pink |
| 83 | 1.04 mmol E + 1.04 mmol F + 0.73 mmol + 0.73 mmol J + 0.42 mmol | yellow- |
| | K + 0.21 mmol L + 0.42 mmol R + 0.42 mmol S | orange |
| 84 | 1.04 mmol E + 1.04 mmol F + 0.73 mmol I + 0.73 mmol J + 0.42 mmol | red-orange |
| | K + 0.21 mmol L + 0.42 mmol T + 0.42 mmol U | |
| 85 | 1.25 mmol E + 1.25 mmol G + 0.75 mmol M + 0.5 mmol N + 0.5 mmol | matt pink, |
| | O + 0.5 mmol P + 0.25 mmol Q | medium |
| 86 | 1.04 mmol E + 1.04 mmol G + 0.73 mmol I + 0.73 mmol J + 0.42 mmol | beige-orange |
| | K + 0.21 mmol L + 0.42 mmol R + 0.42 mmol S | |
| 87 | 1.04 mmol E + 1.04 mmol G + 0.73 mmol I + 0.73 mmol J + 0.42 mmol | beige-pink |
| | K + 0.21 mmol L + 0.42 mmol T + 0.42 mmol U | |
| 88 | 1.25 mmol E + 1.25 mmol H + 0.75 mmol M + 0.5 mmol N + 0.5 mmol | dark blonde |
| | O + 0.5 mmol P + 0.25 mmol Q | pink |
| 89 | 1.04 mmol E + 1.04 mmol H + 0.73 mmol I + 0.73 mmol J + 0.42 mmol | blonde |
| | K + 0.21 mmol L + 0.42 mmol R + 0.42 mmol S | orange |
| 90 | 1.04 mmol E + 1.04 mmol H + 0.73 mmol I + 0.73 mmol J + 0.42 mmol | blue-orange |
| | K + 0.21 mmol L + 0.42 mmol T + 0.42 mmol U | |
| 91 | 1.25 mmol A + 1.25 mmol C + 0.875 mmol I + 0.875 mmol J + 0.5 mmol | red-violet |
| | K + 0.25 mmol L | |
| 92 | 1.04 mmol A + 1.04 mmol C + 0.73 mmol I + 0.73 mmol J + 0.42 mmol | red-brown |
| | K + 0.21 mmol L + 0.42 mmol R + 0.42 mmol S | violet |
| 93 | 1.04 mmol A + 1.04 mmol C + 0.73 mmol I + 0.73 mmol J + 0.42 mmol | red-brown |
| | K + 0.21 mmol L + 0.42 mmol T + 0.42 mmol U | orange |
| 94 | 1.25 mmol B + 1.25 mmol C + 0.875 mmol I + 0.875 mmol J + 0.5 mmol | lilac, dark |
| | K + 0.25 mmol L | |
| 95 | 1.04 mmol B + 1.04 mmol C + 0.73 mmol I + 0.73 mmol J + 0.42 mmol | brown-violet |
| | K + 0.21 mmol L + 0.42 mmol R + 0.42 mmol S | pink |
| 96 | 1.04 mmol B + 1.04 mmol C + 0.73 mmol I + 0.73 mmol J + 0.42 mmol | violet-pink, |
| | K + 0.21 mmol L + 0.42 mmol T + 0.42 mmol U | dark |
| 97 | 1.25 mmol C + 1.25 mmol D + 0.875 mmol I + 0.875 mmol J + 0.5 mmol | red-violet, |
| | K + 0.25 mmol L | pale |
| 98 | 1.04 mmol C + 1.04 mmol D + 0.73 mmol I + 0.73 mmol J + 0.42 mmol | red-brown- |
| | K + 0.21 mmol L + 0.42 mmol R + 0.42 mmol S | violet, pale |
| 99 | 1.04 mmol C + 1.04 mmol D + 0.73 mmol I + 0.73 mmol J + 0.42 mmol | red-brown |
| | K + 0.21 mmol L + 0.42 mmol T + 0.42 mmol U | orange, pale |
| 100 | 1.25 mmol C + 1.25 mmol F + 0.875 mmol I + 0.875 mmol J + 0.5 mmol | red, dark |
| | K + 0.25 mmol L | |
| 101 | 1.04 mmol C + 1.04 mmol F + 0.73 mmol I + 0.73 mmol J + 0.42 mmol | red-orange, |
| | K + 0.21 mmol L + 0.42 mmol R + 0.42 mmol S | medium |
| 102 | 1.04 mmol C + 1.04 mmol F + 0.73 mmol I + 0.73 mmol J + 0.42 mmol | red-yellow, |
| | K + 0.21 mmol L + 0.42 mmol T + 0.42 mmol U | pale |
| 103 | 1.25 mmol C + 1.25 mmol G + 0.875 mmol I + 0.875 mmol | violet-red |
| | J + 0.5 mmol K + 0.25 mmol L | matt |
| 104 | 1.04 mmol C + 1.04 mmol G + 0.73 mmol I + 0.73 mmol J + 0.42 mmol | red-gold |
| | K + 0.21 mmol L + 0.42 mmol R + 0.42 mmol S | matt |
| 105 | 1.04 mmol C + 1.04 mmol G + 0.73 mmol I + 0.73 mmol J + 0.42 mmol | red-orange |
| | K + 0.21 mmol L + 0.42 mmol T + 0.42 mmol U | matt |
| 106 | 1.25 mmol C + 1.25 mmol H + 0.875 mmol I + 0.875 mmol | blue-red |
| | J + 0.5 mmol K + 0.25 mmol L | matt |
| 107 | 1.04 mmol C + 1.04 mmol H + 0.73 mmol I + 0.73 mmol J + 0.42 mmol | blue-gold |
| | K + 0.21 mmol L + 0.42 mmol R + 0.42 mmol S | matt |
| 108 | 1.04 mmol C + 1.04 mmol H + 0.73 mmol I + 0.73 mmol J + 0.42 mmol | blue-orange |
| | K + 0.21 mmol L + 0.42 mmol T + 0.42 mmol U | matt |
| 109 | 1.25 mmol A + 1.25 mmol C + 0.75 mmol M + 0.5 mmol N + 0.5 mmol | red-blue, |
| | O + 0.5 mmol P + 0.25 mmol Q | dark |
| 110 | 1.04 mmol A + 1.04 mmol C + 0.73 mmol I + 0.73 mmol J + 0.42 mmol | red-blue, |
| | K + 0.21 mmol L + 0.42 mmol R + 0.42 mmol S | medium |
| 111 | 1.04 mmol A + 1.04 mmol C + 0.73 mmol I + 0.73 mmol J + 0.42 mmol | red-blue, |
| | K + 0.21 mmol L + 0.42 mmol T + 0.42 mmol U | pale |
| 112 | 1.25 mmol B + 1.25 mmol C + 0.75 mmol M + 0.5 mmol N + 0.5 mmol | red-violet, |
| | O + 0.5 mmol P + 0.25 mmol Q | dark |
| 113 | 1.04 mmol B + 1.04 mmol C + 0.73 mmol I + 0.73 mmol J + 0.42 mmol | brown- |
| | K + 0.21 mmol L + 0.42 mmol R + 0.42 mmol S | violet, |
| | | medium |
| 114 | 1.04 mmol B + 1.04 mmol C + 0.73 mmol I + 0.73 mmol J + 0.42 mmol | brown- |
| | K + 0.21 mmol L + 0.42 mmol T + 0.42 mmol U | violet, pale |
| 115 | 1.25 mmol C + 1.25 mmol D + 0.75 mmol M + 0.5 mmol N + 0.5 mmol | red-blue, |
| | O + 0.5 mmol P + 0.25 mmol Q | medium |
| 116 | 1.04 mmol C + 1.04 mmol D + 0.73 mmol I + 0.73 mmol J + 0.42 mmol | red-blue |
| | K + 0.21 mmol L + 0.42 mmol R + 0.42 mmol S | gold, pale |
| 117 | 1.04 mmol C + 1.04 mmol D + 0.73 mmol I + 0.73 mmol J + 0.42 mmol | red-blue |
| | K + 0.21 mmol L + 0.42 mmol T + 0.42 mmol U | orange, pale |
| 118 | 1.25 mmol C + 1.25 mmol F + 0.75 mmol M + 0.5 mmol N + 0.5 mmol | red-blue, |
| | O + 0.5 mmol P + 0.25 mmol Q | pale |
| 119 | 1.04 mmol C + 1.04 mmol F + 0.73 mmol I + 0.73 mmol J + 0.42 mmol | red-orange, |
| | K + 0.21 mmol L + 0.42 mmol R + 0.42 mmol S | pale |
| 120 | 1.04 mmol C + 1.04 mmol F + 0.73 mmol I + 0.73 mmol J + 0.42 mmol | orange-blue, |
| | K + 0.21 mmol L + 0.42 mmol T + 0.42 mmol U | pale |
| 121 | 1.25 mmol C + 1.25 mmol G + 0.75 mmol M + 0.5 mmol N + 0.5 mmol | red-blue, |
| | O + 0.5 mmol P + 0.25 mmol Q | pale |
| 122 | 1.04 mmol C + 1.04 mmol G + 0.73 mmol I + 0.73 mmol J + 0.42 mmol | red-blue |
| | K + 0.21 mmol L + 0.42 mmol R + 0.42 mmol S | gold, |
| | | medium |
| 123 | 1.04 mmol C + 1.04 mmol G + 0.73 mmol I + 0.73 mmol J + 0.42 mmol | red-blue |
| | K + 0.21 mmol L + 0.42 mmol T + 0.42 mmol U | orange, dark |
| 124 | 1.25 mmol C + 1.25 mmol H + 0.75 mmol M + 0.5 mmol N + 0.5 mmol | red-violet, |
| | O + 0.5 mmol P + 0.25 mmol Q | medium |
| 125 | 1.04 mmol C + 1.04 mmol H + 0.73 mmol I + 0.73 mmol J + 0.42 mmol | red-orange, |
| | K + 0.21 mmol L + 0.42 mmol R + 0.42 mmol S | matt |
| 126 | 1.04 mmol C + 1.04 mmol H + 0.73 mmol I + 0.73 mmol J + 0.42 mmol | red-orange |
| | K + 0.21 mmol L + 0.42 mmol T + 0.42 mmol U | yellow |

### Example 127

### Comparative Experiments Cationic Resin in Anionic and Nonionic Base Mass

Mass I, anionic (not in accordance with the invention)
   7.00 g cetylstearyl alcohol
   2.00 g glyceryl oleate
   3.00 g sodium cetearyl sulfate
   6.00 g PEG-120 methylglucose dioleate
   0.50 g sodium sulfite, anhydrous
   0.10 g ethylenediaminetetraacetic acid disodium salt
   1.20 g polyquaternium-6
   0.80 g 1,4-diamino-2-methylbenzene hydrosulfate
   0.40 g 1,3-dihydroxybenzene
   0.03 g 3-aminophenol
   8.00 g ammonia, 25% strength aqueous solution ad 100.00 g water
Mass II, nonionic (according to the invention)
   7.00 g cetylstearyl alcohol
   2.00 g glyceryl oleate
   13.00 g polysorbate 40
   6.00 g ceteareth-50
   0.50 g sodium sulfite, anhydrous
   0.10 g ethylenediaminetetraacetic acid disodium salt
   1.20 g polyquaternium-6
   0.80 g 1,4-diamino-2-methylbenzene hydrosulfate
   0.40 g 1,3-dihydroxybenzene
   0.03 g 3-aminophenol
   8.00 g ammonia, 25% strength aqueous solution ad 100.00 g water

60 g of the above coloring cream I and 60 g of the coloring cream II are mixed in each case directly prior to application with 60 g of the 6 percent strength hydrogen peroxide emulsion described under Examples 1 to 126. The pH of the resulting ready-to-use composition is about 10. The resulting ready-to-use compositions are each applied to one half of the head hair of a subject divided by a longitudinal parting (half-side test). The ready-to-use composition containing mass I is applied to the left-hand half, and the ready-to-use composition comprising mass II is applied to the right-hand half. After a contact time of 30 minutes at room temperature, they are rinsed out with water. The hair is then washed with a standard commercial shampoo and then dried. The hair is given a medium brown coloration.

The condition of the hair after coloring was assessed by 10 trained hairstyling experts. The feel after washing with shampoo on the left-hand side (not in accordance with the invention) was graded 4, on the right-hand side (according to the invention) 2. On the evaluation scale ranging from 1 to 5, 1 corresponds to a very good feel and 5 to a very poor feel. The comparative experiment shows that the ready-to-use composition prepared with mass II compared with that prepared from mass I has a significantly better and simultaneously significantly greater wash-resistant care effect.

### Example 128

### Skin Compatibility by Means of the Stinging Test

The stinging test is an in vivo method for testing subjective irritant sensations in subjects with sensitive skin. The method is described in more detail in the literature (Stinging Test according to P. Frosch: Hautirritation und empfindliche Haut [skin irritation and sensitive skin], grosse scripta 7, 1985).

The test preparation is applied to a nasolabial fold and the skin sensation is assessed by the subject himself after 10 seconds and 2.5 minutes, 5 minutes and 8 minutes the basis of the following scale:
0 no effect to slight feeling of tautness
1 unpleasant feeling of tautness to slight burning
2 medium strong burning or stinging
3 severe pain

Using this method, the two ready-to-use compositions according to Example 127, both starting from mass I (not in accordance with the invention) and also from mass II according to the invention) were tested. The panel of subjects consisted of 25 stinging-sensitive male and female subjects aged from 18 to 66 years with healthy skin. The results show that, under the test conditions, the ready-to-use composition comprising mass II has a statistically significantly better subjective skin compatibility than that comprising mass I.

### Example 129

### Stability Comparison of Color Masses with any Individual Nonionic Surfactants (not in Accordance with the Invention) and the Combination According to the Invention

Mass III (not in accordance with the invention; pH = 10.5)

| | | |
|---|---|---|
| 8.0 | g | cetylstearyl alcohol |
| 3.0 | g | glyceryl oleate |
| 15.0 | g | PEG-35 castor oil |
| 0.8 | g | polyquaternium-6 |
| 0.5 | g | sodium sulfite, anhydrous |
| 0.1 | g | ethylenediaminetetraacetic acid disodium salt |
| 3.5 | g | 1,4-diamino-2-methylbenzene hydrosulfate |
| 0.8 | g | 1,3-dihydroxybenzene |
| 1.2 | g | 3-aminophenol |
| 8.0 | g | ammonia, 25% strength aqueous solution |
| ad 100.0 | g | water |

Mass IV (not in accordance with the invention; pH = 10.5)

| | | |
|---|---|---|
| 8.0 | g | cetylstearyl alcohol |
| 3.0 | g | glyceryl oleate |
| 15.0 | g | polysorbate-20 |
| 0.8 | g | polyquaternium-6 |
| 0.5 | g | sodium sulfite, anhydrous |
| 0.1 | g | ethylenediaminetetraacetic acid disodium salt |
| 3.5 | g | 1,4-diamino-2-methylbenzene hydrosulfate |
| 0.8 | g | 1,3-dihydroxybenzene |
| 1.2 | g | 3-aminophenol |
| 8.0 | g | ammonia, 25% strength aqueous solution |
| ad 100.0 | g | water |

Mass V (not in accordance with the invention; pH = 10.5)

| | | |
|---|---|---|
| 8.0 | g | cetylstearyl alcohol |
| 3.0 | g | glyceryl oleate |
| 15.0 | g | coceth-10 |
| 0.8 | g | polyquaternium-6 |
| 0.5 | g | sodium sulfite, anhydrous |
| 0.1 | g | ethylenediaminetetraacetic acid disodium salt |
| 3.5 | g | 1,4-diamino-2-methylbenzene hydrosulfate |
| 0.8 | g | 1,3-dihydroxybenzene |
| 1.2 | g | 3-aminophenol |
| 8.0 | g | ammonia, 25% strength aqueous solution |
| ad 100.0 | g | water |

Mass VI (not in accordance with the invention; pH = 10.5)

| | | |
|---|---|---|
| 8.0 | g | cetylstearyl alcohol |
| 3.0 | g | glyceryl oleate |
| 15.0 | g | laneth-40 |
| 0.8 | g | polyquaternium-6 |
| 0.5 | g | sodium sulfite, anhydrous |
| 0.1 | g | ethylenediaminetetraacetic acid disodium salt |
| 3.5 | g | 1,4-diamino-2-methylbenzene hydrosulfate |
| 0.8 | g | 1,3-dihydroxybenzene |
| 1.2 | g | 3-aminophenol |
| 8.0 | g | ammonia, 25% strength aqueous solution |
| ad 100.0 | g | water |

Mass VII (not in accordance with the invention; pH = 10.5)

| | | |
|---|---|---|
| 8.0 | g | cetylstearyl alcohol |
| 3.0 | g | glyceryl oleate |
| 15.0 | g | PPG-38 buteth-37 |
| 0.8 | g | polyquaternium-6 |
| 0.5 | g | sodium sulfite, anhydrous |
| 0.1 | g | ethylenediaminetetraacetic acid disodium salt |
| 3.5 | g | 1,4-diamino-2-methylbenzene hydrosulfate |
| 0.8 | g | 1,3-dihydroxybenzene |
| 1.2 | g | 3-aminophenol |
| 8.0 | g | ammonia, 25% strength aqueous solution |
| ad 100.0 | g | water |

Mass VIII (not in accordance with the invention; pH = 10.5)

| | | |
|---|---|---|
| 8.0 | g | cetylstearyl alcohol |
| 3.0 | g | glyceryl oleate |
| 15.0 | g | polyglyceryl-3 methylglucose distearate |
| 2.0 | g | polyquaternium-6 |
| 0.5 | g | sodium sulfite, anhydrous |
| 0.1 | g | ethylenediaminetetraacetic acid disodium salt |
| 3.5 | g | 1,4-diamino-2-methylbenzene hydrosulfate |
| 0.8 | g | 1,3-dihydroxybenzene |
| 1.2 | g | 3-aminophenol |
| 8.0 | g | ammonia, 25% strength aqueous solution |
| ad 100.0 | g | water |

Mass IX (not in accordance with the invention; pH = 10.5)

| | | |
|---|---|---|
| 8.0 | g | cetylstearyl alcohol |
| 3.0 | g | glyceryl oleate |
| 15.0 | g | polysorbate-40 |
| 0.8 | g | polyquaternium-6 |
| 0.5 | g | sodium sulfite, anhydrous |
| 0.1 | g | ethylenediaminetetraacetic acid disodium salt |
| 3.5 | g | 1,4-diamino-2-methylbenzene hydrosulfate |
| 0.8 | g | 1,3-dihydroxybenzene |
| 1.2 | g | 3-aminophenol |
| 8.0 | g | ammonia, 25% strength aqueous solution |
| ad 100.0 | g | water |

Mass X (not in accordance with the invention; pH = 10.5)

| | | |
|---|---|---|
| 8.0 | g | cetylstearyl alcohol |
| 3.0 | g | glyceryl oleate |
| 15.0 | g | ceteareth-50 |
| 0.8 | g | polyquaternium-6 |
| 0.5 | g | sodium sulfite, anhydrous |
| 0.1 | g | ethylenediaminetetraacetic acid disodium salt |
| 3.5 | g | 1,4-diamino-2-methylbenzene hydrosulfate |
| 0.8 | g | 1,3-dihydroxybenzene |
| 1.2 | g | 3-aminophenol |
| 8.0 | g | ammonia, 25% strength aqueous solution |
| ad 100.0 | g | water |

Mass XI (not in accordance with the invention; pH = 10.5)

| | | |
|---|---|---|
| 8.0 | g | cetylstearyl alcohol |
| 3.0 | g | glyceryl oleate |
| 15.0 | g | PEG-7 glyceryl cocoate |
| 2.0 | g | polyquaternium-6 40% strength solution |
| 0.5 | g | sodium sulfite, anhydrous |
| 0.1 | g | ethylenediaminetetraacetic acid disodium salt |
| 3.5 | g | 1,4-diamino-2-methylbenzene hydrosulfate |
| 0.8 | g | 1,3-dihydroxybenzene |
| 1.2 | g | 3-aminophenol |
| 8.0 | g | ammonia, 25% strength aqueous solution |
| ad 100.0 | g | water |

Mass XII (not in accordance with the invention; pH = 10.5)

| | | |
|---|---|---|
| 8.0 | g | cetylstearyl alcohol |
| 3.0 | g | glyceryl oleate |
| 15.0 | g | laneth-20 |
| 0.8 | g | polyquaternium-6 |
| 0.5 | g | sodium sulfite, anhydrous |
| 0.1 | g | ethylenediaminetetraacetic acid disodium salt |
| 3.5 | g | 1,4-diamino-2-methylbenzene hydrosulfate |
| 0.8 | g | 1,3-dihydroxybenzene |
| 1.2 | g | 3-aminophenol |
| 8.0 | g | ammonia, 25% strength aqueous solution |
| ad 100.0 | g | water |

Mass XIII (not in accordance with the invention; pH = 10.5)

| | | |
|---|---|---|
| 8.0 | g | cetylstearyl alcohol |
| 3.0 | g | glyceryl oleate |
| 15.0 | g | oleth-5 |
| 0.8 | g | polyquaternium-6 |
| 0.5 | g | sodium sulfite, anhydrous |
| 0.1 | g | ethylenediaminetetraacetic acid disodium salt |
| 3.5 | g | 1,4-diamino-2-methylbenzene hydrosulfate |
| 0.8 | g | 1,3-dihydroxybenzene |
| 1.2 | g | 3-aminophenol |
| 8.0 | g | ammonia, 25% strength aqueous solution |
| ad 100.0 | g | water |

Mass XIV (not in accordance with the invention; pH = 10.5)

| | | |
|---|---|---|
| 8.0 | g | cetylstearyl alcohol |
| 3.0 | g | glyceryl oleate |
| 15.0 | g | ceteareth-25 |
| 0.8 | g | polyquaternium-6 |
| 0.5 | g | sodium sulfite, anhydrous |
| 0.1 | g | ethylenediaminetetraacetic acid disodium salt |
| 3.5 | g | 1,4-diamino-2-methylbenzene hydrosulfate |
| 0.8 | g | 1,3-dihydroxybenzene |
| 1.2 | g | 3-aminophenol |
| 8.0 | g | ammonia, 25% strength aqueous solution |
| ad 100.0 | g | water |

Mass XV (not in accordance with the invention; pH = 10.5)

| | | |
|---|---|---|
| 8.0 | g | cetylstearyl alcohol |
| 3.0 | g | glyceryl oleate |
| 15.0 | g | laureth-2 |
| 0.8 | g | polyquaternium-6 |
| 0.5 | g | sodium sulfite, anhydrous |
| 0.1 | g | ethylenediaminetetraacetic acid disodium salt |
| 3.5 | g | 1,4-diamino-2-methylbenzene hydrosulfate |
| 0.8 | g | 1,3-dihydroxybenzene |
| 1.2 | g | 3-aminophenol |
| 8.0 | g | ammonia, 25% strength aqueous solution |
| ad 100.0 | g | water |

Mass XVI (according to the invention; pH = 10.5)

| | | |
|---|---|---|
| 8.0 | g | cetylstearyl alcohol |
| 3.0 | g | glyceryl oleate |
| 13.0 | g | polysorbate-40 |
| 5.0 | g | ceteareth-50 |
| 0.8 | g | polyquaternium-6 |
| 0.5 | g | sodium sulfite, anhydrous |
| 0.1 | g | ethylenediaminetetraacetic acid disodium salt |
| 3.5 | g | 1,4-diamino-2-methylbenzene hydrosulfate |
| 0.8 | g | 1,3-dihydroxybenzene |
| 1.2 | g | 3-aminophenol |
| 8.0 | g | ammonia, 25% strength aqueous solution |
| ad 100.0 | g | water |

Mass XVII (according to the invention; pH = 9.8)

| | | |
|---|---|---|
| 8.0 | g | cetylstearyl alcohol |
| 3.0 | g | glyceryl oleate |
| 13.0 | g | polysorbate-40 |
| 5.0 | g | ceteareth-50 |
| 3.0 | g | cocamide-MEA |
| 1.0 | g | polyglyceryl-2 diisostearate |
| 2.0 | g | polyquaternium-6 40% strength solution |
| 0.5 | g | sodium sulfite, anhydrous |
| 0.1 | g | ethylenediaminetetraacetic acid disodium salt |
| 3.5 | g | 1,4-diamino-2-methylbenzene hydrosulfate |
| 0.8 | g | 1,3-dihydroxybenzene |
| 1.2 | g | 3-aminophenol |
| 2.0 | g | tris(hydroxymethyl)aminomethane |
| 6.0 | g | ammonia, 25% strength aqueous solution |
| ad 100.0 | g | water |

In the case of example masses V and VII to XV, no cream-like emulsions can be formed by simple stirring. Although masses III, IV and VI form cream-like emulsions by simple stirring, these separate again after storage for a few hours at 40 degrees Celsius. Only through the combination according to the invention of selected nonionic emulsions according to the example masses XVI and XVII is it possible to prepare, by simple stirring, stable cream-like emulsions which are storage-stable over several months at 40 degrees Celsius and do not separate into an aqueous phase and an oily phase.

### Example 130

### Oxidation Hair Colorant, Cream-Like

20 g of the color carrier masses XVI and XVII from Example 129 are mixed directly prior to application with 20 g of the 6 percent strength hydrogen peroxide emulsion described in Examples 1 to 126. The pH of the resulting ready-to-use composition is in each case about 10. The mixture obtained in this way is applied in each case to bleached hair tresses and, after a contact time of 30 minutes at room temperature, rinsed out with water. The hair is then washed with a standard commercial shampoo and then dried. The hair is in each case given a black coloration.

Unless stated otherwise, all of the percentages given in the present application are percentages by weight.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An emulsion-like dye carrier mass comprising (a) water, (b) oxidation color precursors and optionally additionally direct dyes and (c) customary cosmetic cationic polymers, characterized in that it comprises (d) at least one nonionic organic thickener with wax-like properties selected from the group consisting of C12-C24-fatty alcohols, glycerol oleate and mixtures thereof, (e) as nonionic surfactant a mixture of at least one sorbitan fatty acid ester oxyethylated with about 4 to about 20 mol of ethylene oxide and at least one C12-C24-fatty alcohol oxyethylated with about 30 to about 70 mol of ethylene oxide, and (f) being free from anionic and cationic surfactants.

2. A dye carrier mass according to claim 1, characterized in that said nonionic organic thickener with wax-like properties is selected from the group consisting of cetyl alcohol, stearyl alcohol, glyceryl monooleate and mixtures thereof.

3. A dye carrier mass according to claim 1, characterized in that said nonionic surfactant is chosen from mixtures of (i) sorbitan lauric esters ethoxylated with about 20 mol of ethylene oxide, sorbitan palmitic esters ethoxylated with about 20 mol of ethylene oxide or sorbitan stearic esters ethoxylated with about 20 mol of ethylene oxide and (ii) cetyl alcohol ethoxylated with about 30 to about 70 mol of ethylene oxide, stearyl alcohol ethoxylated with about 30 to about 70 mol of ethylene oxide or cetylstearyl alcohol ethoxylated with about 30 to about 70 mol of ethylene oxide.

4. A dye carrier mass according to claim 3, characterized in that said nonionic surfactant is chosen from a mixture of a sorbitan palmitic ester ethoxylated with about 20 mol of ethylene oxide and a cetylstearyl alcohol ethoxylated with about 50 mol of ethylene oxide.

5. A dye carrier mass according to claim 1, characterized in that said nonionic organic thickener with wax-like properties is used in a total amount of from about 5 to about 25 percent by weight.

6. A dye carrier mass according to claim 1, characterized in that said nonionic surfactant is used in a total amount of from about 3 to about 25 percent by weight.

7. A dye carrier mass according to claim 1, characterized in that said cationic polymer is selected from the group consisting of Polyquaternium-6, Polyquaternium-7, Polyquaternium-10, Polyquaternium-11 and Polyquaternium-35, and mixtures thereof.

8. A dye carrier mass according to claim 1, characterized in that said cationic polymer is used in a total amount of from about 0.1 to about 6 percent by weight.

9. A two-component composition for the oxidative coloring of hair consisting of two components (A) and (B) to be mixed directly prior to application, characterized in that the component (A) is a dye carrier mass comprising (a) water, (b) oxidation color precursors and optionally additionally direct dyes, (c) customary cosmetic cationic polymers, (d) at least one nonionic organic thickener with wax-like properties from the group consisting of C12-C24-fatty alcohols and glycerol oleate and mixtures thereof, and (e) as nonionic surfactant a mixture of at least one sorbitan fatty acid ester oxyethylated with about 4 to about 20 mol of ethylene oxide and at least one C12-C24-fatty alcohol oxyethylated with from about 30 to about 70 mol of ethylene oxide, and (f) being free from anionic and cationic surfactants, and the component (B) is a liquid or cream-like oxidizing agent preparation with a content of from about 1 to about 12 percent by weight of an oxidizing agent.

10. A ready-to-use composition for the oxidative coloring of hair which is obtained by mixing a color carrier mass (A) comprising (a) water, (b) oxidation color precursors and optionally additionally direct dyes, (c) customary cosmetic cationic polymers, (d) at least one nonionic organic thickener with wax-like properties from the group consisting of C12-C24-fatty alcohols and glycerol oleate and mixtures thereof, and (e) as nonionic surfactant a mixture of at least one sorbitan fatty acid ester oxyethylated with about 4 to about 20 mol of ethylene oxide and at least one C12-C24-fatty alcohol oxyethylated with about 30 to about 70 mol of ethylene oxide, and (f) being free from anionic and cationic surfactants, and a preparation (B) comprising about 1 to about 12 percent by weight of an oxidizing agent, in the ratio (A):(B) of from about 5:1 to about 1:4.
